(19) United States
(12) Patent Application Publication
(10) Pub. No.: US 2024/0398877 A1
(43) Pub. Date: Dec. 5, 2024

(54) LACTOBACILLUS STRAIN USABLE FOR STIMULATING AND REBALANCING INTESTINAL MICROBIOTA

(71) Applicant: ALMA MATER STUDIORUM - Universita' di Bologna, BOLOGNA (IT)

(72) Inventors: Beatrice Vitali, San Lazzaro di Savena (BO) (IT); Carola Eleonora Parolin, Casalecchio di Reno (BO) (IT); Barbara Giordani, Bologna (BO) (IT); Barbara Luppi, Villanova di Castenaso (BO) (IT); Angela Abruzzo, Bologna (BO) (IT); Antonella Marangoni, San Lazzaro di Savena (BO) (IT); Claudio Foschi, Forli' (FC) (IT)

(73) Assignee: ALMA MATER STUDIORUM - Universita' di Bologna, BOLOGNA (IT)

(21) Appl. No.: 18/700,724

(22) PCT Filed: Oct. 13, 2022
(86) PCT No.: PCT/IB2022/059819

§ 371 Date: Apr. 12, 2024

(30) Foreign Application Priority Data
Oct. 14, 2021 (IT) 102021000026339

(51) Int. Cl. A61K 35/742; A61K 9/00; A61K 9/06

(52) U.S. Cl. CPC A61K 35/742; A61K 9/0014; A61K 9/006; A61K 9/06

(57) ABSTRACT

The Limosilactobacillus vaginalis BC17 strain, deposited with number DSM 34059, is usable as an agent for promoting a proliferation of intestinal bifidobacteria in a human subject. A method for promoting the proliferation of intestinal bifidobacteria in a human subject provides for administering to the human subject the Limosilactobacillus vaginalis BC17 strain deposited with number DSM 34059. The strain can be incorporated into a liquid or solid composition that can be administered orally or buccally and the human subject can be a neonate or a suckling. The strain can also be used to prepare compositions that are applicable to the skin of the nipples of a breast-feeding woman.

The invention relates to a lactobacillus strain usable for stimulating and/or rebalancing the intestinal microbiota, in particular the intestinal microbiota of neonates and sucklings.

A healthy intestinal microbiota, in particular an intestinal microbiota that is rich in bifidobacteria, promotes a correct development and a correct maturation of the immune system, as well as prevents chronic, allergic and auto-immune inflammatory pathologies. On the contrary, changes to the microbial profile are considered to be the main causes of severe gastrointestinal infections during infancy and the development of chronic, allergic and auto-immune inflammatory pathologies (Rutayisire E, Huang K, Liu Y, Tao F. The mode of delivery affects the diversity and colonization pattern of the gut microbiota during the first year of infants' life: a systematic review. BMC Gastroenterol. 2016; 16 (1): 86. doi: 10.1186/s12876-016-0498-0. PMID: 27475754; PMCID: PMC4967522; Dominguez-Bello M G, Costello E K, Contreras M, Magris M, Hidalgo G, Fierer N, Knight R. Delivery mode shapes the acquisition and structure of the initial microbiota across multiple body habitats in newborns. Proc Natl Acad Sci USA. 2010; 107 (26): 11971-5. doi: 10.1073/pnas. 1002601107. PMID: 20566857; PMCID: PMC2900693; Chu D M, Ma J, Prince A L, Antony K M, Seferovic M D, Aagaard K M. Maturation of the infant microbiome community structure and function across multiple body sites and in relation to mode of delivery. Nat Med. 2017; 23 (3): 314-326. doi: 10.1038/nm.4272. PMID: 28112736; PMCID: PMC5345907; Francino M P. Birth Mode-Related Differences in Gut Microbiota Colonization and Immune System Development. Ann Nutr Metab. 2018; 73 Suppl 3:12-16. doi: 10.1159/000490842. PMID: 30041189).

A drawback observed in neonates delivered by caesarean section is a drastically lower presence of bifidobacteria as compared, for example, spontaneously delivered neonates. This increases the possibility that these neonates will develop gastrointestinal disorders, such as diarrhoea, irritable bowel syndrome and colics. Also in the case of artificially fed infants, the development of an intestinal microbiota that is more deficient of Bifidobacterium strains than in breast-fed infants can be observed.

Therefore, among the skilled in the art persons (neonatologists and paediatric nutritionists) a strong need is felt for new means usable to suitably treat a possible deficiency of bifidobacteria in the intestinal microbiota of neonates and sucklings.

### OBJECTS OF THE INVENTION

An object of the invention is to provide a composition for promoting the proliferation of intestinal bifidobacteria in neonates, in particular neonates born by caesarean section, and in sucklings, in particular artificially fed sucklings.

Another object is to provide a method for promoting the proliferation of intestinal bifidobacteria in neonates, in particular neonates born by caesarean section, and in sucklings, in particular artificially fed sucklings.

### SHORT DESCRIPTION OF THE INVENTION

In a first aspect of the invention, a lactobacillus strain is provided for use as an agent for promoting proliferation of intestinal bifidobacteria in a human subject, as defined in claim 1.

In a second aspect of the invention, a method is provided for promoting a proliferation of intestinal bifidobacteria in a human subject, as defined in claim 19.

Owing to these aspects, a lactobacillus strain that is able to effectively promote the proliferation of intestinal bifidobacteria and a method that comprises administering the aforesaid strain to a human subject, in particular a neonate or suckling, are made available.

The strain according to the invention is incorporable into a composition, which is in turn administrable orally to a neonate or a suckling and in particular to a neonate born by caesarean section or to an artificially fed suckling. Moreover, the strain according to the invention is usable for preparing a composition for topical use, which can be applied to the skin of the nipples of a breast-feeding woman. This enables at the same time to prevent the appearance of rhagades on the breast (regenerative and soothing action) and to promote the balance of the intestinal bacterial flora of the sucklings (stimulation of the proliferation of bifidobacteria, following ingestion of the active component during suckling).

In one embodiment, the composition comprises, as an active component, a supernatant recovered from a culture of the strain according to the invention. The supernatant is devoid of bacterial cells, but it contains metabolites that are produced by the lactobacillus and have a bifidogenic action.

In another embodiment, the composition comprises, as an active component, live bacterial cells of the strain according to the invention, which bacterial cells, suitably transported, can transitionally colonize the intestine and promote the growth of the bifidobacteria.

In a further embodiment, the composition is an oil- or water-based oral liquid composition.

In another further embodiment, the composition is a solid composition for oral or buccal administration.

In a still further embodiment, the composition is a semi-solid composition, which is applicable to the skin of the nipples of a breast-feeding woman. The semi-solid composition can be oil-based or water-based and can contain the supernatant recovered from a culture of the strain according to the invention, together with substances having a soothing and regenerative action.

In a still further embodiment, the aforesaid semi-solid composition contains bacterial cells inactivated by known procedures (thermally or by radiations) of the strain according to the invention.

### SHORT DESCRIPTION OF THE DRAWINGS

FIG. 1 shows the stimulation effects produced by supernatants of Limosilactobacillus vaginalis BC17 cultivated for 7 hours (top diagram), 13 hours (intermediate diagram) and 24 hours (bottom diagram) towards planktonic cultures of bifidobacteria.

FIG. 2 shows the stimulation effects produced by supernatants of Limosilactobacillus vaginalis BC17 cultivated for 7 hours (top diagram), 13 hours (intermediate diagram) and 24 hours (bottom diagram) towards the formation of bifidobacteria biofilm.

### DETAILED DESCRIPTION OF THE INVENTION

Lactobacilli are microorganisms that distinguish the human vaginal microbiote; bacteria belonging to the Lactobacillus genus are in fact abundant and dominant in the vaginal environment of healthy women of reproductive age (Ravel J, Gajer P, Abdo Z, Schneider G M, Koenig S S, McCulle S L, Karlebach S, Gorle R, Russell J, Tacket C O, Brotman R M, Davis C C, Ault K, Peralta L, Forney L J. Vaginal microbiome of reproductive-age women. Proc Natl Acad Sci USA. 2011 Mar. 15; 108 Suppl 1:4680-7. doi: 10.1073/pnas. 1002611107. PMID: 20534435; PMCID: PMC3063603). It is widely demonstrated that vaginal lactobacilli are involved in maintaining the state of vaginal eubiosis, protecting the female genital tract from microbial dysbiosis, inflammatory states and sexually transmitted infections.

The Inventors have identified and verified experimentally a new and unexpected effect, namely the bifidogenic activity exhibited by the Limosilactobacillus vaginalis BC17 strain (indicated below also more concisely as the “L. vaginalis BC17” or “BC17”). The BC17 strain was isolated from the vaginal mucosa of a woman of child-bearing age (Parolin C, et al. (2015) Isolation of Vaginal Lactobacilli and Characterization of Anti-Candida Activity. PLOS ONE 10 (6): e0131220) and deposited on Oct. 6, 2021 at the DSMZ collection (German Collection of Microorganisms and Cell Cultures, Inhoffenstr. 7B, 38124 Braunschweig, https://www.dsmz.de) with number DSM 34059.

In particular, the bifidogenic activity of BC17 was tested by the Inventors towards 11 strains of Bifidobacterium deriving from the DSMZ collection and belonging to species that are very common in the human intestine: B. longum subsp. infantis DSM20090, B. longum subsp. infantis DSM20088, B. longum subsp. longum DSM20219, B. bifidum DSM20082, B. bifidum DSM20215, B. bifidum DSM20213, B. breve DSM20091, B. breve DSM20456, B. adolescentis DSM20086, B. adolescentis DSM20083, B. angulatum DSM20098.

BC17 was cultivated in a de Man, Rogosa and Sharpe liquid medium (MRS) (Beckton, Dickinson and Co., Milan, Italy) added to 0.05% (w/v) L-cysteine (Sigma-Aldrich, Milan, Italy) at 37° C. in an anaerobiosis jar containing GasPak EZ (Beckton, Dickinson and Co.).

At different incubation times (after 7, 13 and 24 hours), 10 ml of bacterial culture were centrifuged (10000 g, 10 minutes). The culture-medium supernatants containing the metabolites produced by BC17 were filtered (0.22 μm PES filters for syringe, VWR International, Milan, Italy), taken to pH=6 with NaOH 5M to simulate the pH of the intestinal tract and used for the bifidobacteria stimulation tests.

The Bifidobacterium strains were cultivated in anaerobic conditions in MRS medium for 24 hours and subsequently diluted in medium to obtain cellular suspensions (106 CFU/ml) to be used as an inoculum. The supernatants of BC17 recovered after 7, 13 and 24 hours were diluted in MRS medium inside multi-well plates (96 wells) with a U-shaped bottom. The ½, ¼ and ⅛ dilutions of the original samples were tested. 100 μl of bifidobacterium suspension were inoculated together with 100 μl of sample (diluted supernatant). The growth control for each bifidobacterium consisted of the cell suspension (100 μl) plus MRS (100 μl).

The multi-well plates were incubated in anaerobiosis (37° C. in an anaerobiosis jar containing GasPak EZ) and the effect of the BC17 supernatants on the planktonic cultures of the bifidobacteria and on the formation of biofilm of the bifidobacteria was evaluated.

The growth effect on the planktonic cultures (free growth in a liquid medium) was evaluated after 24 hours of incubation by reading (apparatus: Empire Multimode Plate Reader) of the optical density (or absorbance) at 600 nm (OD600).

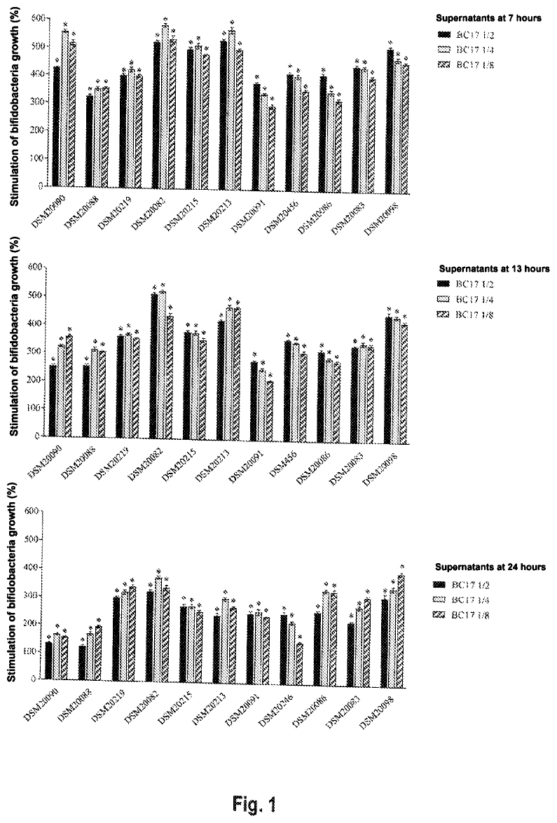

The results are shown in FIG. 1 as a growth percentage of the bifidobacteria in the presence of the supernatants with respect to the control (100%), according to the equation (Eq.1):

$$Stimulation of bifidobacteria growth ( \% ) = ( OD 600 sample / OD 600 control ) \times 100 \qquad ( Eq . 1 )$$

In FIG. 1, the top diagram illustrates the effects of the BC17 supernatant cultivated for 7 hours, the central diagram illustrates the effects of the BC17 supernatant cultivated for 13 hours and the bottom diagram illustrates the effects of the BC17 supernatant cultivated for 24 hours. In all the diagrams, the asterisk (*) indicates a significant difference from the growth control (ANOVA with Bonferroni correction, p<0.05).

The growth effect on the formation of bifidobacteria biofilm was evaluated using the procedure described below.

After 48 hours of incubation, the supernatants were removed and the adherent cells were washed with physiological solution and fixed with 200 μl of 96% ethanol for 5 minutes. The cells were then coloured with 200 μl of crystal violet 0.41% (w/v) for 5 minutes. Once the dye was removed, the wells were washed three times with sterile water. Lastly, the dye bound to the cells was resolubilized with 200 μl of 96% ethanol and the optical density at 595 nm (OD595) was measured.

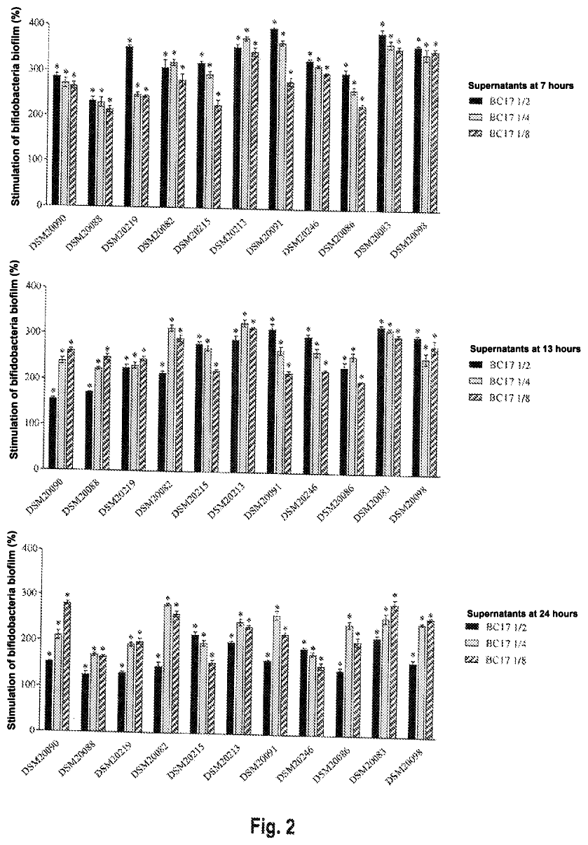

The activity exerted by the BC17 supernatants on the biofilms is illustrated by the diagrams of FIG. 2, in which it was expressed as a percentage with respect to the control (100%), according to the equation (Eq. 2):

$$Stimulation of bifidobacteria biofilm ( \% ) = ( OD 595 sample / OD 595 control ) \times 100 \qquad ( Eq . 2 )$$

In FIG. 2, the top diagram illustrates the effects of the BC17 supernatant cultivated for 7 hours, the central diagram illustrates the effects of the BC17 supernatant cultivated for 13 hours and the bottom diagram illustrates the effects of the BC17 supernatant cultivated for 24 hours. In all the diagrams, the asterisk (*) indicates a significant difference from the growth control (ANOVA with Bonferroni correction, p<0.05).

It has thus been demonstrated that BC17 shows a bifidogenic activity towards all the Bifidobacterium strains tested (FIG. 1). In fact, the BC17 supernatant recovered after only 7 hours of fermentation is able to stimulate the planktonic growth of the bifidobacteria in percentages varying between 324% and 523% at the ½ dilution. The stimulating activity is widely clear also at the greater dilutions (342-570% at the ¼ dilution and 299-536% at the ⅛ dilution), indicating that the metabolites produced by BC17 maintain the ability to exert a bifidogenic action also at lower concentrations.

A good stimulating activity was also observed with the BC17 supernatant deriving from longer fermentation periods, with stimulation percentages of 250-512% (supernatant recovered at 13 hours) and of 122-323% (supernatant at 24 hours), at the ½ dilution. Also in this case the bifidogenic action is maintained at lower concentrations of supernatant, confirming the previous datum.

The effect of the BC17 strain on the production of the biofilms by the strains of Bifidobacterium was evaluated because the capacity to form biofilms is an important factor for the profitable colonization of the bifidobacteria at the intestinal level. The obtained results (FIG. 2) show that the BC17 supernatant, in addition to stimulating the proliferation of the bifidobacteria, is also able to promote the adhesion and formation of the biofilm by the bifidobacteria.

Consistently with what has been observed for the planktonic cultures, the greatest stimulation was obtained with the supernatant recovered after 7 hours of fermentation (stimulation of the biofilm of 232-396% at the ½ dilution) and the activity is maintained even following dilution of the sample.

The obtained in vitro results indicate that long fermentation times of BC17 are not necessary to obtain a supernatant having the desired functional properties, which constitutes a significant technical advantage for the purposes of a pharmaceutical production on an industrial scale. This further suggests that in vivo, for example following an oral administration as a supplement, BC17 is able to perform a bifidogenic activity for long periods in the human organism.

In one embodiment of the invention, oil-based or water-based oral liquid compositions are provided containing, as an active main component, live bacterial cells of the Limosilactobacillus vaginalis BC17 strain or supernatant recovered from cultures of the Limosilactobacillus vaginalis BC17 strain and another possible active component consisting of one or more vitamins that are useful for the growth and development of children.

The vitamins can comprise at least one of the following: vitamin A (contributing to the normal metabolism of iron, the maintenance of visual capacity and mucosae, the normal function of the immune system), vitamin B9 (contributing to the normal function of the immune system), vitamin B12 (promoting the normal functions of the nervous and immune systems), vitamin C (contributing to the normal formation of the collagen for the function of the blood vessels, the bones, the cartilage, the gums, the teeth, to the energetic metabolism and the function of the nervous and immune systems, as well as to the protection of the cells from oxidative stress), vitamin D3 (promoting the normal absorption and use of calcium and phosphorus) and vitamin E (protecting the cells from oxidative stress).

The oral liquid compositions according to the invention can further contain excipients of known type, such as lipid bases (oil bases), sweetening agents, flavouring agents, emulsifying agents for food use and preservative agents for food use.

The lipid bases are provided in the compositions in quantities that go from 70% w/w to 90% w/w and can comprise vegetable oils and/or oils of synthetic origin.

The vegetable oils can comprise at least one of the following: sweet almond oil, sunflower seed oil, linseed oil, wheat germ oil, maize oil, rice oil, olive oil, avocado oil, jojoba oil.

The oils of synthetic origin can comprise medium-chain triglycerides and/or vitamin E acetate.

The sweetening agents are provided in the compositions in quantities that go from 0.1% w/w to 20% w/w and can comprise at least one of the following: glucose, fructose, dextrose, sucrose, sorbitol, maltitol, mannitol, saccharin.

The flavouring agents are provided in the compositions in quantities that go from 0.1% w/w to 5% w/w and can comprise at least one of the following: strawberry flavour, orange flavour, wild berries flavour.

The emulsifying agents for food use are provided in the compositions in quantities that go from 0.5% w/w to 10% w/w and can comprise E472 and/or E433.

The preservative agents for food use are provided in the compositions in quantities that go from 0.5% w/w to 2% w/w and can comprise E202.

In another embodiment of the invention, solid oral or buccal compositions (namely, solid compositions for oral or buccal administration) are provided containing, as a main active component, live bacterial cells of the Limosilactobacillus vaginalis BC17 strain or supernatant recovered from cultures of the Limosilactobacillus vaginalis BC17 strain and a possible other active component consisting of one or more vitamins used for the growth and development of children and/or one or more prebiotics.

The dosage form of the solid compositions according to the invention is a tablet, which is produced through lyophilization by using known apparatuses and methods.

In one embodiment, the tablet can be applied on the tongue of a subject (oral administration) and is intended for a rapid release (less than a minute without the need to take liquids) at the level of the oral cavity. In this embodiment, highly soluble excipients of known type are selected and used.

In another embodiment, the tablet can be applied on the gum of a subject (buccal administration) and is intended for a sustained release (up to a maximum of 4-6 hours). In this embodiment, excipients of known type are selected and used that are able to gel and to promote adhesion of the tablet to the gum.

The vitamins comprise at least one of the following: vitamin A, vitamin B9, vitamin B12, vitamin C, vitamin D3 and vitamin E.

The prebiotics are provided in the compositions in quantities that go from 1% w/w to 10% w/w and can comprise fructo-oligosaccharides and/or skimmed milk.

The solid (oral or buccal) compositions according to the invention can further contain excipients of known type, such as polymers, sweetening agents, cryoprotective agents, salivating agents, adsorbent agents, emulsifying agents for food use and preservative agents for food use.

The polymers are provided in the compositions in quantities that go from 0.5% w/w to 2% w/w and can comprise at least one of the following: starch, gums, pullulan, alginates, hyaluronates, carrageenins, pectin, gelatin, maltodextrin, hydroxypropyl cellulose, hydroxypropyl methyl cellulose, polyvinyl alcohol, polyvinylpyrrolidone.

The sweetening agents are provided in the compositions in quantities that go from 0.1% w/w to 20% w/w and can comprise at least one of the following: glucose, fructose, dextrose, sucrose, sorbitol, maltitol, mannitol, saccharin.

The cryoprotective agents are provided in the compositions in quantities that go from 0.1% w/w to 10% w/w and can comprise at least one of the following: glucose, sucrose, trehalose, lactose, fructose, mannitol, skimmed milk.

The salivating agents are provided in the compositions in quantities that go from 0.1% to 10% w/w and can comprise at least one of the following: citric acid, lactic acid, malic acid, ascorbic acid.

The adsorbing agents are provided in the compositions in quantities that go from 0.1% to 20% w/w and can comprise at least one of the following: microcrystalline cellulose, talcum, micronized silicon, kaolin.

The flavouring agents are provided in the compositions in quantities that go from 0.1% w/w to 5% w/w and can comprise at least one of the following: strawberry flavour, orange flavour, wild berries flavour.

The emulsifying agents for food use are provided in the compositions in quantities that go from 0.5% w/w to 10% w/w and can comprise E472 and/or E433.

The preservative agents for food use are provided in the compositions in quantities that go from 0.5% w/w to 2% w/w and can comprise E202.

In a further embodiment of the invention, compositions for topical use are provided, more exactly oil-based or water-based semi-solid compositions containing, as a main active component, inactivated bacterial cells of the Limosilactobacillus vaginalis BC17 strain or supernatant recovered from cultures of the Limosilactobacillus vaginalis BC17 strain. The semi-solid compositions according to the invention can be adsorbed on suitable supports of known type, such as for example gauze compresses and pads, which are intended for being applied on the skin of the nipples of a lactating woman. The semi-solid compositions according to the invention can comprise another possible active component, consisting of one or more substances having soothing and regenerative action, such as for example vitamin E, hyaluronic acid.

The oil-based or water-based semi-solid compositions can further contain excipients of known type, such as lipid bases (oil bases), polymers, gelling agents, wetting agents, sweetening agents, emulsifying agents for food use and preservative agents for food use.

The lipid bases are provided in the compositions in quantities that go from 70% w/w to 90% w/w and can comprise vegetable oils and/or oils of synthetic origin.

The vegetable oils can comprise at least one of the following: sweet almond oil, sunflower seed oil, linseed oil, wheat germ oil, maize oil, rice oil, olive oil, avocado oil, jojoba oil.

The oils of synthetic origin can comprise at least one of the following: lanolin, medium-chain triglycerides, vitamin E acetate.

The polymers are provided in the compositions in quantities that go from 1% w/w to 4% w/w and can comprise at least one of the following: starch, gums, pullulan, alginates, hyaluronates, carrageenins, pectin, gelatin, maltodextrin, hydroxypropyl cellulose, hydroxypropyl methyl cellulose, polyvinyl alcohol, polyvinylpyrrolidone.

The gelling agents are provided in the compositions in quantities that go from 2% w/w to 10% w/w and can comprise at least one of the following: micronized silicon, aluminium soaps, zinc soaps.

The wetting agents are provided in the compositions in quantities that go from 0.5% w/w to 3% w/w and can comprise at least one of the following: polyalkylene oxides, glycerol, monoacetate glycerol, propylene glycol and PEG. The polyalkylene oxides can comprise polyethylene glycol and/or polypropylene glycol.

The sweetening agents are provided in the compositions in quantities that go from 0.1% w/w to 20% w/w and can comprise at least one of the following: glucose, fructose, dextrose, sucrose, sorbitol, maltitol, mannitol, saccharin.

The flavouring agents are provided in the compositions in quantities that go from 0.1% w/w to 5% w/w and can comprise at least one of the following: strawberry flavour, orange flavour, wild berries flavour.

The emulsifying agents for food use are provided in the compositions in quantities that go from 0.5% w/w to 10% w/w and can comprise E472 and/or E 433.

The preservative agents for food use are provided in the compositions in quantities that go from 0.5% w/w to 2% w/w and can comprise E202.

By way of non-limiting example of the invention, the following Examples are disclosed below: liquid composition containing live bacterial cells of the strain according to the invention (Example 1); solid composition containing live bacterial cells of the strain according to the invention (Example 2); solid composition containing supernatant recovered from cultures of the strain according to the invention (Example 3); semi-solid composition containing supernatant recovered from cultures of the strain according to the invention (Example 4); semi-solid composition containing inactivated bacterial cells of the strain according to the invention (Example 5).

### Example 1—Liquid Composition Containing Live Bacterial Cells of the Limosilactobacillus vaginalis BC17 Strain

An oil-based suspension was prepared containing live bacterial cells of the L. vaginalis BC17 strain and the vitamins of interest. The aforesaid suspension can be taken as such in drops or be dispersed in water, milk or other food liquids.

Functional ingredients (active components): L. vaginalis BC17 (10⁸-10⁹ cfu/dose—5 drops), vitamin D3 oil (10 mcg/dose—5 drops).

Excipients: sunflower seed oil (oil base).

The vitamin D3 (Farmalabor, Canosa di Puglia, Italy; 0.01% w/w) was mixed with the sunflower seed oil (ACEF, Piacenza, Italy; 89.99% w/w) under stirring at 300 rpm for 1 hour. Subsequently, previously lyophilized (0.01 atm, −45° C.; Christ Freeze Dryer ALPHA 1 and 2, Milan, Italy) L. vaginalis BC17 was dispersed in the mixture at the desired concentration.

### Example 2—Solid Composition Containing Live Bacterial Cells of the Limosilactobacillus vaginalis BC17 Strain

The solid composition according to the invention was prepared in an oral dosage form and in a buccal dosage form.

### Oral Composition-Rapid Release Lyophilized Tablets

An oral composition was prepared containing live bacterial cells of the L. vaginalis BC17 strain and the vitamins of interest. The dosage form is a tablet obtained through lyophilization. The tablet is applicable on the tongue of a subject and is able to disintegrate and release rapidly the components thereof without the need to use water.

Functional ingredients (active principles): L. vaginalis BC17 (10⁸-10⁹ cfu/dose), vitamin D3 100 (10 mcg/dose).

Excipients: gelatin (polymer), mannitol (sweetener and cryoprotective), fructo-oligosaccharides (prebiotic and cryoprotective), ascorbic acid (salivating and antioxidant agent), skimmed milk (prebiotic and cryoprotective).

The gelatin (Sigma Aldrich, Milan, Italy; 1.5% w/w) was solubilized in sterile FU purified water at 50° C. for 1 hour. Subsequently, mannitol (ACEF, Piacenza, Italy; 1% w/w), fructo-oligosaccharides (Farmalabor, Canosa di Puglia, Italy, 10% w/w), ascorbic acid (Sigma Aldrich, Milan, Italy; 2.5% w/w), skimmed milk (Sigma Aldrich, Milan, Italy; 2.5% w/w) and vitamin D3 100 (Farmalabor, Canosa di Puglia, Italy, 0.002% w/w) that had been previously dispersed in water at 35-40° C. were added. The solution was left under stirring for 3 hours at 300 rpm. Subsequently, L. vaginalis BC17 was added to the solution at the desired concentration. Lastly, 0.5 g of the so obtained suspension were inserted inside each cavity of a blister for tablets (diameter 13 mm; Farmalabor, Canosa di Puglia, Italy). The blisters were frozen for 24 hours at −20° C. and lyophilized (0.01 atm, −45° C.; Christ Freeze Dryer ALPHA 1 and 2, Milan, Italy) for 24 hours.

### Buccal Composition-Sustained Release Lyophilized Tablets

An oral composition containing live bacterial cells of the L. vaginalis BC17 strain and the vitamins of interest was prepared. The dosage form is a tablet obtained through lyophilization. The tablet is applicable on the gum of a subject and is able to gel and gradually release the components thereof (sustained release).

Functional ingredients (active principles): L. vaginalis BC17 (10⁸-10⁹ cfu/dose), vitamin D3 100 (10 mcg/dose).

Excipients: hydroxypropyl methyl cellulose (polymer), mannitol (sweetener and cryoprotective), fructo-oligosaccharides (prebiotic and cryoprotective), ascorbic acid (salivating and antioxidant agent), skimmed milk (prebiotic and cryoprotective).

The hydroxypropyl methyl cellulose (ASHLAND, Switzerland; 1.5% w/w) was solubilized in water for 24 hours. Subsequently, mannitol (ACEF, Piacenza, Italy; 1% w/w), fructo-oligosaccharides (Farmalabor, Canosa di Puglia, Italy, 10% w/w), ascorbic acid (Sigma Aldrich, Milan, Italy; 2.5% w/w), skimmed milk (Sigma Aldrich, Milan, Italy; 2.5% w/w) and vitamin D3 100 (Farmalabor, Canosa di Puglia, Italy, 0.002% w/w) that had been previously dispersed in water at 35-40° C. were added. The solution was left under stirring for 3 hours at 300 rpm. Subsequently, the L. vaginalis BC17 was added at the desired concentration. Lastly, 0.5 g of the so obtained suspension were inserted inside each cavity of a blister for tablets (diameter 13 mm; Farmalabor, Canosa di Puglia, Italy). The blisters were frozen for 24 hours at −20° C. and lyophilized (0.01 atm, −45° C.; Christ Freeze Dryer ALPHA 1 and 2, Milan, Italy) per 24 hours.

### Example 3—Solid Composition Containing Supernatant Recovered from Cultures of the Limosilactobacillus vaginalis BC17 Strain

The solid composition according to the invention was prepared in an oral dosage and in a buccal dosage form.

### Oral Composition-Rapid Release Lyophilized Tablets

An oral composition containing supernatant recovered from cultures of the L. vaginalis BC17 strain and the vitamins of interest was prepared. The dosage form is a tablet obtained through lyophilization. The tablet is applicable on the tongue of a subject and is able to disintegrate and release rapidly the components thereof without the need to use water.

Functional ingredients (active principles): supernatant recovered from cultures of the L. vaginalis BC17 strain (50 mg/dose), vitamin D3 100 (10 mcg/dose).

Excipients: gelatin (polymer), mannitol (sweetener and cryoprotective), microcrystalline cellulose (adsorbent).

The gelatin (Sigma Aldrich, Milan, Italy; 1.5% w/w) was solubilized in sterile FU purified water at 50° C. for 1 hour. Subsequently, mannitol (ACEF, Piacenza, Italy; 1% w/w), microcrystalline cellulose (Farmalabor, Canosa di Puglia, Italy, 10% w/w) and vitamin D3 100 (Farmalabor, Canosa di Puglia, Italy, 0.002% w/w) that had been previously dispersed in water at 35-40° C. were added. The suspension was left under stirring for 3 hours at 300 rpm. Subsequently, the supernatant recovered from cultures of the L. vaginalis BC17 strain was added at the desired concentration. Lastly, 0.5 g of the so obtained suspension were inserted inside each cavity of a blister for tablets (diameter 13 mm; Farmalabor, Canosa di Puglia, Italy). The blisters were frozen for 24 hours at −20° C. and lyophilized (0.01 atm, −45° C.; Christ Freeze Dryer ALPHA 1 and 2, Milan, Italy) for 24 hours.

### Buccal Composition-Sustained Release Lyophilized Tablets

An oral composition containing supernatant recovered from cultures of the L. vaginalis BC17 strain and the vitamins of interest was prepared. The dosage form is a tablet obtained through lyophilization. The tablet is applicable on the gum of a subject and is able to gel and gradually release the components thereof (sustained release).

Functional ingredients (active principles): supernatant recovered from cultures of the L. vaginalis BC17 strain (50 mg/dose), vitamin D3 100 (10 mcg/dose).

Excipients: hydroxypropyl methyl cellulose (polymer), mannitol (sweetener and cryoprotective), microcrystalline cellulose (adsorbent).

The hydroxypropyl methyl cellulose (ASHLAND, Switzerland; 1.5% w/w) was solubilized in water for 24 hours. Subsequently mannitol (ACEF, Piacenza, Italy; 1% w/w), microcrystalline cellulose (Farmalabor, Canosa di Puglia, Italy, 10% w/w) and vitamin D3 100 (Farmalabor, Canosa di Puglia, Italy, 0.002% w/w) that had been previously dispersed in water at 35-40° C. were added. The suspension was left under stirring for 3 hours at 300 rpm. Subsequently, the supernatant recovered from cultures of the L. vaginalis BC17 strain was added at the desired concentration. Lastly, 0.5 g of the so obtained suspension were inserted inside each cavity of a blister for tablets (diameter 13 mm; Farmalabor, Canosa di Puglia, Italy). The blisters were frozen for 24 hours at −20° C. and lyophilized (0.01 atm, −45° C.; Christ Freeze Dryer ALPHA 1 and 2, Milan, Italy) for 24 hours.

### Example 4—Semi-Solid Composition Containing Supernatant Recovered from Cultures of the Limosilactobacillus vaginalis BC17 Strain

A water-based semi-solid composition was prepared containing supernatant recovered from cultures of the L. vaginalis BC17 strain. The composition according to the invention was prepared in the form of hyaluronic acid-based hydrogel (substance provided with hydrating, ri-epithelizing, soothing, anti-reddening action) obtained by dispersing the functional components in the gelled base.

Functional ingredients (active principles): supernatant recovered from cultures of the L. vaginalis BC17 strain (200 mg/g).

Water base: water, propylene glycol (wetting agent), sodium hyaluronate, potassium sorbate (E202 preservative).

The sodium hyaluronate (Farmalabor, Canosa di Puglia, Italy) was solubilized in water (2% w/w) at 250 rpm for 24 hours. Subsequently, propylene glycol (ACEF, Piacenza, Italy; 1% w/w), potassium sorbate (ACEF, Piacenza, Italy; 1% w/v) were added under stirring (300 rpm) for 30 minutes. In the so obtained gel, the supernatant recovered from cultures of the previously lyophilized (0.01 atm, −45° C.; Christ Freeze Dryer ALPHA 1 and 2, Milan, Italy) L. vaginalis BC17 strain was dispersed at the desired concentration.

### Example 5—Semi-Solid Composition Containing Inactivated Bacterial Cells of the Limosilactobacillus vaginalis BC17 Strain

The composition according to the invention was prepared in the form of an ointment, lipogel and hydrogel.

### Oil-Based Composition—Ointment

An oil-based composition was prepared containing inactivated bacterial cells of the L. vaginalis BC17 strain. The composition is a highly refined lanolin-based ointment.

The aforesaid lanolin has a strong hydrating action, owing to the composition thereof that is rich in esters of higher fatty acids and promotes the formation of a thin protective lipid layer, which is obtained by dispersing functional components in the base.

Functional ingredients (active principles): inactivated L. vaginalis BC17 (100 mg/g).

Oil base: lanolin

The lanolin (ACEF, Piacenza, Italy) was mixed with previously lyophilized (0.01 atm, −45° C.; Christ Freeze Dryer ALPHA 1 and 2, Milan, Italy) inactivated bacterial cells of the L. vaginalis BC17 strain at the desired concentration.

### Oil-Based Composition—Lipogel

An oil-based composition was prepared containing inactivated bacterial cells of the L. vaginalis BC17 strain. The composition is a sweet almond oil/vitamin E acetate-based lipogel, obtained by dispersing the functional components in the gelled base.

The sweet almond oil has an emollient, anti-reddening and elasticizing action, whilst the vitamin E acetate has an itch-soothing, hydrating, protective and antioxidant action.

Functional ingredients (active principles): inactivated L. vaginalis BC17 (100 mg/g).

Oil base: sweet almond oil, vitamin E acetate, micronized silicon (gelling agent).

The sweet almond oil (ACEF, Piacenza, Italy; 40% w/w) was gelled by using micronized silicon (ACEF, Piacenza, Italy; 3.5% w/w) under stirring at 250 rpm per 2 hours. The obtained gel was subsequently mixed with vitamin E acetate (ACEF, Piacenza, Italy; 46.5% w/w) for 30 minutes at 250 rpm. Lastly, previously lyophilized (0.01 atm, −45° C.; Christ Freeze Dryer ALPHA 1 and 2, Milan, Italy) inactivated bacterial cells of the L. vaginalis BC17 strain at the desired concentration were dispersed in the gel.

### Water-Based Composition—Hydrogel

A water-based composition containing inactivated bacterial cells of the L. vaginalis BC17 strain was prepared. The composition is a hyaluronic acid-based hydrogel (substance provided with hydrating, re-epithelizing, soothing, anti-reddening action) obtained by dispersing the functional components in the gelled base.

Functional ingredients (active principles): inactivated L. vaginalis BC17 (100 mg/g).

Water base: water, propylene glycol, sodium hyaluronate, potassium sorbate (E202 preservative).

The sodium hyaluronate (Farmalabor, Canosa di Puglia, Italy) was solubilized in water (2% w/w) at 250 rpm for 24 hours. Subsequently, propylene glycol (ACEF, Piacenza, Italy; 1% w/w), potassium sorbate (ACEF, Piacenza, Italy; 1% p/v) were added under stirring (300 rpm) for 30 minutes. In the so obtained gel, previously lyophilized (0.01 atm, −45° C.; Christ Freeze Dryer ALPHA 1 and 2, Milan, Italy) inactivated bacterial cells of the L. vaginalis BC17 strain were finally dispersed at the desired concentration.

The composition in liquid form according to the invention, as disclosed in Example 1, can be administered as such or dispersed in food liquids to a human subject (neonate or suckling).

The composition in solid form according to the invention, as described in Examples 2 and 3, can be a rapid release composition, which is administered by placing a single tablet directly on the tongue of a neonate or suckling (oral administration), or can be a sustained release composition, which is applied to the gum of the neonate or suckling (buccal administration).

The semi-solid composition described in Examples 4 and 5 is able to perform a regenerating and soothing action, preventing the appearance of rhagades on the breast and at the same time is able to promote the balance of the intestinal bacterial flora of sucklings, stimulating the proliferation of bifidobacteria. The semi-solid composition according to the invention can be adsorbed on a suitable support of known type, such as gauzes, gauze compresses and pads. The support can thus be applied to the nipples of the breast-feeding mother.

From what has been disclosed and exemplified above, it can be stated that the compositions according to the invention, containing the Limosilactobacillus vaginalis BC17 strain, and the method according to the invention enable the set objects to be achieved, namely they are able to promote the proliferation of intestinal bifidobacteria in neonates and sucklings, in particular in neonates born by caesarean section and in artificially fed sucklings.

Variations and/or additions to what have been disclosed above are possible. For example, although the previously disclosed compositions were prepared on a laboratory scale, the skilled in the art person is able to select and apply preparation procedures that are suitable for a production on an industrial scale.

1. Method for promoting a proliferation of intestinal bifidobacterial in a human subject, said method comprising
administering to said human subject Limosilactobacillus vaginalis BC17 strain deposited with number DSM 34059, said human subject comprising a suckling or a neonate.

2. Composition containing the strain according to claim 1.

3. The composition according to claim 2, comprising live bacterial cells of said strain or supernatant recovered from cultures of said strain.

4. The composition according to claim 3, wherein said composition is a liquid composition.

5. The liquid composition according to claim 4, wherein said liquid composition can be administered orally.

6. The liquid composition according to claim 4, further comprising at least one vitamin selected from the group consisting of: vitamin A, vitamin B9, vitamin B12, vitamin C, vitamin D3, vitamin E and mixtures thereof.

7. The composition according to claim 3, wherein said composition is a solid composition.

8. The solid composition according to claim 7, wherein said live bacterial cells or said supernatant are in a lyophilized form.

9. The solid composition according to claim 7 or 8, where said solid composition can be administered orally or buccally.

10. The solid composition according to claim 7, wherein said solid composition is in the form of a rapid release tablet for oral administration.

11. The solid composition according to claim 7, wherein said solid composition is in the form of a sustained release tablet for buccal administration.

12. The solid composition according to claim 7, further comprising at least one vitamin selected from the group consisting of: vitamin A, vitamin B9, vitamin B12, vitamin C, vitamin D3, vitamin E and mixtures thereof.

13. The composition according to claim 2, wherein said composition is a composition for topical use and comprises inactivated bacterial cells of said strain or supernatant recovered from cultures of said strain.

14. The composition according to claim 13, wherein said composition for topical use is a semi-solid composition.

15. The semi-solid Semi-composition according to claim 14, wherein said semi-solid composition is in a form selected from the group consisting of: ointment, lipogel and hydrogel.

16. The composition according to claim 13, further comprising at least one substance having a soothing and regenerating action.

17. The composition according to claim 16, wherein said at least one substance having a soothing and regenerating action is selected from the group consisting of: E vitamin, hyaluronic acid and mixtures thereof.

18. The composition according to claim 13, wherein said topical use comprises an application of said composition on the skin of the nipples of a breast-feeding woman.

19. (canceled)
